(19) United States
(12) Patent Application Publication
(10) Pub. No.: US 2025/0356990 A1
(43) Pub. Date: Nov. 20, 2025

(54) PERCUTANEOUS CORONARY INTERVENTION PLANNING

(71) Applicant: Medtronic Vascular, Inc., Santa Rosa, CA (US)

(72) Inventors: Stephen Nash, Athlone (IE); Paul J. Coates, Corte Madera, CA (US); Patrick A. Helm, Canton, MA (US); Darion R. Peterson, Longmont, CO (US)

(21) Appl. No.: 18/871,090

(22) PCT Filed: Jun. 6, 2023
(86) PCT No.: PCT/US2023/024598

§ 371 Date: Dec. 2, 2024

Related U.S. Application Data
(60) Provisional application No. 63/365,932, filed on Jun. 6, 2022.

(51) Int. Cl. G16H 30/20; G16H 10/60; G16H 15/00; G16H 20/10; G16H 20/40; G16H 40/20; G16H 40/60

(52) U.S. Cl. CPC G16H 30/20; G16H 10/60; G16H 15/00; G16H 20/10; G16H 20/40; G16H 40/20; G16H 40/60

(57) ABSTRACT

Example medical systems and techniques are disclosed. A medical device system includes memory configured to store one or more procedural plans and processing circuitry communicatively coupled to the memory. The processing circuitry is configured to receive pre-therapeutic imaging data, the pre-therapeutic imaging data being indicative of a coronary issue in at least a portion of a vasculature of a patient. The processing circuitry is configured to automatically determine, based on the pre-therapeutic imaging data, a procedural plan for use during a therapeutic medical procedure in a catheter laboratory (Cath Lab) and output the procedural plan.

This application claims the benefit of U.S. Provisional Application No. 63/365,932, filed Jun. 6, 2022, and entitled, “PERCUTANEOUS CORONARY INTERVENTION PLANNING.”

### TECHNICAL FIELD

This disclosure relates to the planning and assisting of a medical procedure.

### BACKGROUND

A percutaneous coronary intervention (PCI) is a medical procedure used to address coronary issues, such as lesions within a vasculature of a patient. Such procedures may be performed in a Catheterization Laboratory (Cath Lab) and may include inserting a catheter into the vasculature of the patient to implant a stent, inflate a balloon, remove calcification, and/or the like. A Cath Lab is a specialized facility, which may be located in a hospital, that includes cardiac imaging equipment. The cardiac imaging equipment may be used by a clinician to diagnose a cardiac issue of the patient and/or to assist the clinician in visualizing the vasculature of the patient during a therapeutic medical procedure such as a PCI to treat a cardiac issue of the patient. Such an imaging system may display anatomy, medical instruments, or the like, and may be used to diagnose a patient condition or assist in guiding a clinician in moving a medical instrument to an intended location inside the patient. Imaging systems may use sensors to capture video images which may be displayed during the medical procedure. Imaging systems include angiography systems, ultrasound imaging systems, computed tomography (CT) scan systems, magnetic resonance imaging (MRI) systems, isocentric C-arm fluoroscopic systems, positron emission tomography (PET) systems, intravascular ultrasound (IVUS), optical coherence tomography (OCT), as well as other imaging systems.

### SUMMARY

In general, this disclosure is directed to various techniques and medical systems for planning medical procedures and updating medical plans during procedures. This disclosure is also related to various techniques for training machine learning algorithms and/or artificial intelligence algorithms which may be used when planning such medical procedures and/or updating the plans for such medical procedures.

Currently, noninvasive coronary imaging data is predominantly used for diagnosing the coronary issue(s) and not for a medical procedure such as a PCI. While there are planning tools that are aimed at facilitating a clinician to use the noninvasive image and to plan a medical procedure such as a PCI, these plans may not currently integrate with the Cath Lab where the PCI may be performed.

According to the techniques of this disclosure, a medical system may use a trained machine learning algorithm and/or an artificial intelligence algorithm to plan a medical procedure, such as a PCI procedure, based on data collected prior to the medical procedure. Such data may include noninvasive imaging data, invasive imaging data, and/or sensor data. For example, the medical system may generate a procedural plan which may be displayed or otherwise presented to a clinician both before the medical procedure and during the medical procedure to assist the clinician in performing the procedure. During the medical procedure additional data may be collected and such data may be used by processing circuitry executing the trained machine learning algorithm and/or the trained artificial intelligence algorithm to determine that a different or additional treatment may be more likely to yield a better outcome for the patient than a treatment that is in the original procedural plan. In such a case, the processing circuitry may update the procedural plan to include the different or additional treatment. The machine learning algorithm and/or an artificial intelligence algorithm may be trained on a combination of pre-procedural data, intra-procedural data, and post-procedural data.

By using a trained machine learning algorithm and/or a trained artificial intelligence algorithm to generate a procedural plan and/or update the procedural plan during a medical procedure, patient outcomes may be improved, resulting in better health for the patient post-procedure.

Aspects of this disclosure are applicable to at least Cath Lab procedures. Example Cath Lab procedures include, but are not necessarily limited to, coronary procedures, renal denervation (RDN) procedures, structural heart and aortic (SH&A) procedures (e.g., transcatheter aortic valve replacement (TAVR), transcatheter mitral valve replacement (TMVR), and the like), device implantation procedures (e.g., heart monitors, pacemakers, defibrillators, and the like), etc.

In one example, the disclosure describes a medical system comprising memory configured to store one or more procedural plans; and processing circuitry communicatively coupled to the memory, the processing circuitry being configured to: receive pre-therapeutic imaging data, the pre-therapeutic imaging data being indicative of a coronary issue in at least a portion of a vasculature of a patient; automatically determine, based at least in part on the pre-therapeutic imaging data, a procedural plan for use during a therapeutic medical procedure in a Catheterization Laboratory; and output the procedural plan.

In another example, the disclosure describes a method comprising receiving, by processing circuitry, procedural pre-therapeutic imaging data, the pre-therapeutic imaging data being indicative of a coronary issue in at least a portion of a vasculature of a patient; automatically determining, by the processing circuitry and based at least in part on the pre-therapeutic imaging data, a procedural plan for use during a therapeutic medical procedure in a Catheterization Laboratory; and outputting, by the processing circuitry, the procedural plan.

In yet another example, the disclosure describes a non-transitory computer readable medium comprising instructions, which, when executed, cause processing circuitry to receive pre-therapeutic imaging data, the pre-therapeutic imaging data being indicative of a coronary issue in at least a portion of a vasculature of a patient; automatically determine, based at least in part on the pre-therapeutic imaging data, a procedural plan for use during a therapeutic medical procedure in a Catheterization Laboratory; and output the procedural plan.

These and other aspects of the present disclosure will be apparent from the detailed description below. In no event, however, should the above summaries be construed as limitations on the claimed subject matter, which subject matter is defined solely by the attached claims.

This summary is intended to provide an overview of the subject matter described in this disclosure. It is not intended to provide an exclusive or exhaustive explanation of the apparatus and methods described in detail within the accompanying drawings and description below. Further details of one or more examples are set forth in the accompanying drawings and the description below.

### BRIEF DESCRIPTION OF DRAWINGS

FIG. 1 is a schematic perspective view of one example of a system for guiding a medical instrument through a region of a patient.

FIG. 2 is a schematic view of one example of a computing system of the system of FIG. 1.

FIG. 3 is a functional block diagram illustrating an example system that includes remote computing devices, such as a server and one or more other computing devices, that are connected via a network.

FIG. 4 is a flow diagram of example generation of a procedural plan techniques according to one or more aspects of this disclosure.

FIG. 5 is a flow diagram of example machine learning algorithm or artificial intelligence algorithm training techniques according to one or more aspects of this disclosure.

FIG. 6 is a conceptual diagram illustrating an example machine learning model according to one or more aspects of this disclosure.

FIG. 7 is a conceptual diagram illustrating an example training process for a machine learning model in accordance with one or more aspects of this disclosure.

### DETAILED DESCRIPTION

Imaging systems may be used to assist a clinician in diagnosing a medical condition, such as a coronary issue, during a medical procedure, such as a percutaneous coronary intervention (PCI) procedure, or both. For example, imaging systems may be used to determine presence of lesions within a vasculature of a patient that may be limiting or obstructing blood flow within the vasculature of the patient. For example, such imaging systems may be used to identify possible coronary issues, including lesions such as bifurcation lesions, calcified lesions, chronic total occlusions (CTOs), in-stent restenosis (ISR), left main disease, etc. Imaging systems may also be used when performing a PCI, such as an angioplasty procedure, or other medical procedure intended to treat lesions within the vasculature of the patient. While described primarily herein with respect to the vasculature of a patient, imaging systems described herein may be used for other medical purposes and are not limited to coronary purposes. Imaging systems may generate static image data or video data via sensors. This data may be recorded for later use. The data may include representations of portions of vasculature of a patient, including one or more lesions which may be restricting blood flow through the portion of the vasculature, a geometry and location within a blood vessel of such lesions, and/or any medical instrument which may be within a field of view of one or more sensors of the imaging system.

As referred to herein, a medical procedure may be a diagnostic medical procedure or a therapeutic medical procedure. A diagnostic medical procedure is a medical procedure in which imaging or other techniques are used to diagnose disease. A therapeutic medical procedure is a medical procedure in which therapy is delivered and/or an intervention is performed, for example, a PCI. A single Cath Lab session may include 1) only a diagnostic medical procedure, for example, where no lesion is identified that requires treatment or in which the treatment is too difficult for a given clinician or the hospital in which the Cath Lab is located does not have the necessary equipment to treat the lesion; 2) only a therapeutic medical procedure, for example, where a lesion was previously diagnosed; or 3) a diagnostic medical procedure followed by a therapeutic medical procedure. As disclosed herein, pre-therapeutic imaging data taken prior to a therapeutic medical procedure, such as a PCI, may be used by a medical system to determine a procedural plan. The medical system may determine the procedural plan through the use of a trained machine learning algorithm and/or a trained artificial intelligence algorithm by inputting pre-procedural data, such as the pre-therapeutic imaging data, into the trained machine learning algorithm and/or a trained artificial intelligence algorithm. For example, the trained machine learning algorithm and/or a trained artificial intelligence algorithm may be trained on pre-procedural data (e.g., pre-therapeutic imaging data), intra-procedural data (e.g., additional imaging data, which may or may not be invasive), and post-procedural data. Differences between the pre-procedural and post-procedural data may be indicative of an outcome of a therapeutic medical procedure. The data used to train the machine learning algorithm and/or the artificial intelligence algorithm may include data from a plurality of patients which have undergone such therapeutic medical procedures.

The procedural plan may be used by a clinician during the therapeutic medical procedure to assist the clinician with the therapeutic medical procedure. Data collected during the therapeutic medical procedure (e.g., intra-procedural data) may be also input into the trained machine learning algorithm and/or a trained artificial intelligence algorithm to determine whether the procedural plan should be updated to include a different treatment not contained within the procedural plan. For example, if the medical system executing the trained machine learning algorithm and/or a trained artificial intelligence algorithm determines that the likelihood of a more successful outcome would be higher if a different or additional treatment would be conducted, the medical system may update the procedural plan to include the different or additional treatment. By generating a procedural plan, the techniques of this disclosure may assist a clinician in performing a procedure. By updating the procedural plan, the techniques of this disclosure may increase a likelihood of a successful outcome for the patient. By training a machine learning algorithm and/or an artificial intelligence algorithm as discussed herein, the procedural plans and updates to the procedural plans may be improved, which may further increase the likelihood of a successful outcome for patients over time.

Thus, techniques of this disclosure bring pre-therapeutic imaging to the planning stage and also integrate the procedural plan with the Cath Lab and the medical instruments or devices used in the Cath Lab by providing a clinician with real time guidance and/or feedback and a record of the therapeutic medical procedure. The overall procedural plan, record of the therapeutic medical procedure, treatments used, and outcome (e.g., determined by the differences between the pre-procedural data and the post-procedural data) may be used as input to a machine learning algorithm and/or an artificial intelligence algorithm to train the machine learning algorithm and/or an artificial intelligence algorithm, which may be used for future procedure planning. Such trained machine learning algorithms and/or artificial intelligence algorithms may be particularly useful for complex PCI of which bifurcation lesions, calcified lesions, CTO, and ISR are subsets.

The techniques of this disclosure bring the pre-procedural data into the Cath Lab and may augment this pre-procedural data with real time data being acquired in the lab. The techniques also allow for the procedural plan to act as a map over which the completed treatment can be overlayed. All this data may be processed by processing circuitry executing a machine learning or artificial intelligence algorithm that can begin to predict outcomes from building a database of plans, treatments, and outcomes for coronary interventions and training the machine learning or artificial intelligence algorithm on such data.

The techniques of this disclosure may be powered by real world data as more therapeutic medical procedures are performed, thus improving the recommendations of treatment. Also, the recommendations may stay up to date with evolving or new techniques and new and existing medical devices because the machine learning algorithm or artificial intelligence algorithm may be further trained on more recent PCI procedures.

Not all clinicians may be comfortable with performing a complex PCI, such as a PCI on a bifurcation case, a calcified lesion case, a CTO case, an ISR case, a left main disease case, etc. However, the procedural plan generated through the techniques of this disclosure may help the clinician plan such a complex case, giving them a starting point for their procedural strategy.

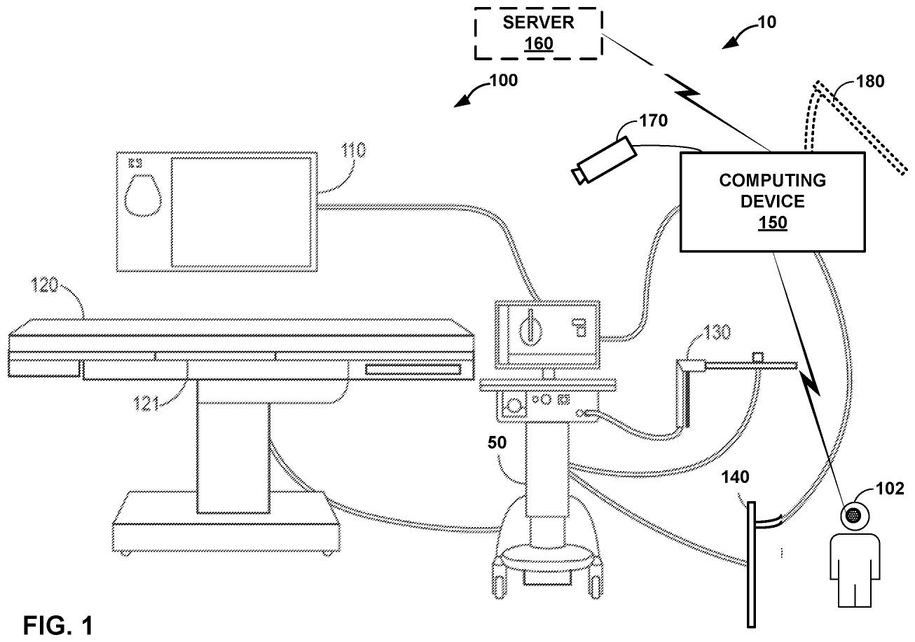

FIG. 1 is a schematic perspective view of one example of a system for guiding a medical instrument through a region of a patient. System 10, at least a portion of which may be in Cath Lab 100, which includes a guidance workstation 50, a display device 110, a table 120, a medical instrument 130, an imager 140, and a computing device 150. Prior to conducting a therapeutic medical procedure, such as a PCI, in Cath Lab 100, a clinician may perform pre-therapeutic imaging of the patient to diagnose a coronary disease. The clinician may also take or receive sensor data from a wearable device (such as a smart watch, fitness watch, or the like), an implantable device, or other sensors, such as a stethoscope, which may be in the office of the clinician. The sensor data may be indicative of a coronary issue. A clinician may also utilize one or more physiological indices (such as fractional flow reserve (FFR), coronary flow reserve (CFR), instantaneous wave-free ratio (iFR), or other flow reserve measure) to identify a coronary issue, such as a significant lesion, including a bifurcation lesion, a calcified lesion, a CTO, an ISR, left main disease, etc. Once a coronary issue is identified, a clinician may determine to perform a therapeutic medical procedure, for example, in Cath Lab 100, to address the coronary issue.

Guidance workstation 50 may include, for example, an off-the-shelf device, such as a laptop computer, desktop computer, tablet computer, smart phone, or other similar device. In some examples, guidance workstation may be a specific purpose device. Guidance workstation 50 may be configured to control an electrosurgical generator, a peristaltic pump, a power supply, or any other accessories and peripheral devices relating to, or forming part of, system 10. Computing device 150 may include, for example, an off-the-shelf device such as a laptop computer, desktop computer, tablet computer, smart phone, or other similar device or may include a specific purpose device.

Display device 110 may be configured to output instructions, images, and messages relating to at least one of a performance, position, orientation, or trajectory of medical instrument 130, coronary anatomy, patient parameters, etc. Display device 110 may also be configured to display a procedural plan. In some examples, display device 110 may display a procedural plan and imaging data collected during a therapeutic medical procedure together at the same time. For example, display device 110 may fuse images in the plan or otherwise taken pre-procedure (e.g., pre-therapeutic imaging data) with real time images taken during the therapeutic medical procedure (e.g., fluoroscopy images, IVUS, OCT, etc.) and provide a three-dimensional (3D) image or side-by-side perspective of anatomy of the patient and device(s) relative to the plan. For example, processing circuitry (e.g., of computing device 150) may overlay or integrate coronary computed tomography angiography (CCTA) images (collected prior to a Cath Lab session) with angiography images collected during a Cath Lab session. For example, the plan may include strategies, medical instruments, and/or devices represented in a graphical or video form to facilitate a clinician in conducting the therapeutic medical procedure. In some examples, processing circuitry may track devices through the use of sensor(s) or by auto image segmentation. Placement of such devices may be compared to the plan. Fusion of pre-PCI images with real time imaging (fluoroscopy, ultrasound, IVUS, OCT, etc.) provides a 3D or side-by-side perspective of anatomy and device(s) relative to the plan.

In some examples, processing circuitry may be configured to share live case data with colleagues for collaboration on treatment strategies. For example, processing circuitry may be configured to control telemetry circuitry to transmit live case data, such as images, treatment plan, etc., to one or more colleagues for display on a mobile device, a tablet, a laptop computer, a desktop computer, a workstation, or the like.

Further, the display device 110 may be configured to output information regarding medical instrument 130, e.g., algorithm number, type, size, etc. Table 120 may be, for example, an operating table or other table suitable for use during a medical procedure that may optionally include an electromagnetic (EM) field generator 121. EM field generator 121 may be optionally included and used to generate an EM field during the medical procedure and, when included, may form part of an EM tracking system that is used to track the positions of one or more medical instruments within the body of a patient. EM field generator 121 may include various components, such as a specially designed pad to be placed under, or integrated into, an operating table or patient bed.

Medical instrument 130 may also be visualized by using imaging, such as angiography (e.g., contrast-enhanced coronary angiography), OCT, or intravascular ultrasound (IVUS) imaging. In the example of FIG. 1, an imager 140, such as an angiography device, may be used to image vasculature of a patient during the medical procedure to visualize the vasculature of the patient, locations of medical instruments, such as surgical instruments, device delivery or placement devices, and implants, inside the patient's body. While described primarily as an angiography imager, imager 140 may be any type of imaging device including one or more sensors.

Imager 140 may image a region of interest in the patient's body. The particular region of interest may be dependent on anatomy, the diagnostic procedure, and/or the intended therapy. For example, when performing a PCI, a portion of the vasculature may be the region of interest.

As described further herein, imager 140 may be positioned in relation to medical instrument 130 such that the medical instrument is at an angle to the image plane, thereby enabling the clinician to visualize the spatial relationship of medical instrument 130 with the ultrasound image plane and with objects being imaged. In some examples, if provided, the EM tracking system may also track the location of imager 140. In one or more examples, imager 140 may be placed inside the body, such as inside the vasculature, of the patient. The EM tracking system may then track the locations of such imager 140 and the medical instrument 130 inside the body of the patient. In some examples, the functions of computing device 150 may be performed by guidance workstation 50 and computing device 150 may not be present.

The location of the medical instrument within the body of the patient may be tracked during the surgical procedure. An exemplary technique of tracking the location of the medical instrument includes using imager 140. Another exemplary technique of tracking the location of the medical instrument includes using the EM tracking system, which tracks the location of medical instrument 130 by tracking sensors attached to or incorporated in medical instrument 130. Prior to starting the medical procedure, the clinician may verify the accuracy of the tracking system using any suitable technique or techniques. Any suitable medical instrument 130 may be utilized with the system 10. Examples of medical instruments or devices include stents, catheters (including guide catheters, guide extension catheters, balloon catheters, etc.), angioplasty devices, atherectomy devices, etc.

Computing device 150 may be communicatively coupled to imager 140, workstation 50, display device 110 and/or server 160, for example, by wired, optical, or wireless communications. Server 160 may be a hospital server, a cloud-based server, or the like. Server 160 may be configured to store a trained machine learning algorithm, a trained artificial intelligence algorithm, patient imaging data, electronic healthcare or medical records, type of coronary issue, severity of the coronary issue, complexity of the coronary issue, location of the coronary issue, classification of a lesion, anatomy in the area of the coronary issue, other anatomy, or the like. In some examples, server 160 may further store patient metadata, such as sex, age, weight, height, body mass index, body fat percentage, comorbidities, cholesterol level, blood pressure, blood oxygenation, physical exercise level, heart rate, or the like. In some examples, computing device 150 may be an example of workstation 50.

Computing device 150 may be configured to receive imaging data from imager 140. Computing device 150 may be configured to share the imaging data with server 160 such that server 160 may execute the trained machine learning algorithm and/or the trained artificial intelligence algorithm to determine whether to update the procedural plan which may be displayed on display device 110. In other examples, computing device 150 may execute the trained machine learning algorithm and/or the trained artificial intelligence algorithm locally to determine whether to update the procedural plan. Data gathered during the therapeutic medical procedure, such as angiographic images, OCT, or intravascular ultrasound, etc., may provide more detailed anatomical and/or physiological data (e.g., FFR or other flow reserve measure, vulnerable plaque identification, etc.) than pre-therapeutic imaging data taken pre-procedure. This data may be added to any records of the overall therapeutic medical procedure and may be used update the treatment strategies.

Computing device 150 may also be configured to present a user interface on a display, such as a display of computing device 150 or display device 110. Such a user interface may be configured to display the procedural plan and intra-procedural imaging data collected by imager 140 so as to guide a clinician performing the therapeutic medical procedure.

Computing device 150 may also be configured to receive imaging data from more than one type of imaging system. For example, imager 140 may be an angiography imager while imager 180 may be fluoroscopy imager. Thus, computing device 150 may receive a plurality of different imaging data. In some examples, computing device 150 may register the plurality of different imaging data and overlay the plurality of imaging data. In some examples, computing device 150 may overlay any of the imaging data being collected during the therapeutic medical procedure with the procedural plan.

Computing device 150 may be configured to receive video data captured by one or more video cameras 170. While only a single video camera is shown, it is to be understood that one or more video cameras 170 may include a plurality of video cameras which may be located in different locations in Cath Lab 100. One or more video cameras 170 may capture video data that includes, for example, hand movements, such as those of a clinician performing the therapeutic medical procedure, robot movements, such as those of robot 102 involved in or performing therapeutic the medical procedure, medical instruments, or devices (e.g., implantable devices) used, when the medical instruments or devices are used, and/or where the medical instruments or devices are used. In some examples, such video data may be used to train the machine learning algorithm and/or artificial intelligence algorithm and/or be used as input to the machine learning algorithm and/or artificial intelligence algorithm when determining whether to update the procedural plan.

In some examples, the machine learning application or the artificial intelligence application may be used with a robotic or robotic-assisted PCI procedure. For example, the robot 102 may be programmed to follow a procedural plan determined or updated by the machine learning application or the artificial intelligence application. While depicted as an android, it should be understood that a robotic arm which may be located near operating table 120 may perform such robotic or robotic-assisted PCI procedure. Machine vision may be used to facilitate robot 102 following the plan based on imaging technologies used intra procedure and/or the video being captured one or more video cameras. The use of robotics, such as robot 102, may result in lower patient and clinician radiation exposure as procedure times may be reduced and/or, for robotic assisted procedures, the clinician may be located remotely from the patient. In a robotic assisted scenario, computing device may include the ability for a clinician to provide input to control the therapeutic medical procedure or to select options. For example, a clinician may interface with a user interface, such as a joystick, a touch screen, a mouse, or the like, and control the movement of a guide wire by the robotics to desired location. In another example, the clinician may select a location on the imaging, such as by touching or clicking on the location and the robotics may deliver a device to that location. In some examples, the robotics may provide feedback as the robotics delivers the device to the location, such as imaging feedback.

Computing device 150 may be configured to upload any data collected during the therapeutic medical procedure to server 160. Computing device 150 may also be configured to generate a report for a clinician or the patient including data collected during the therapeutic medical procedure. In some examples, computing device 150 may upload the report to server 160. Computing device 150 and/or server 160 may be configured to update the report to generate an updated report based on post-procedural data relating to the patient. For example, such post-procedural data may include post-procedural sensor data relating to the patient, user input data relating to the patient, post-procedural imaging data of the patient, or physiological data of the patient. Post-procedural sensor data may include data from a wearable device, such as a smart watch or fitness watch, such as heartrate data, oxygenation data, quantity of steps taken, or the like. Differences between post-procedural data and pre-procedural data may be indicative of an outcome of the therapeutic medical procedure. User input data may include data input by the clinician or the patient, such as how the patient is feeling, how much exercise the patient is getting, other sensor data, for example, that is sensed during a post-procedural office visit, or the like. Post-procedural imaging data may include pre-therapeutic imaging data taken during a post-procedural office visit. Physiological data may include, for example, an FFR or other flow reserve analysis, or the like.

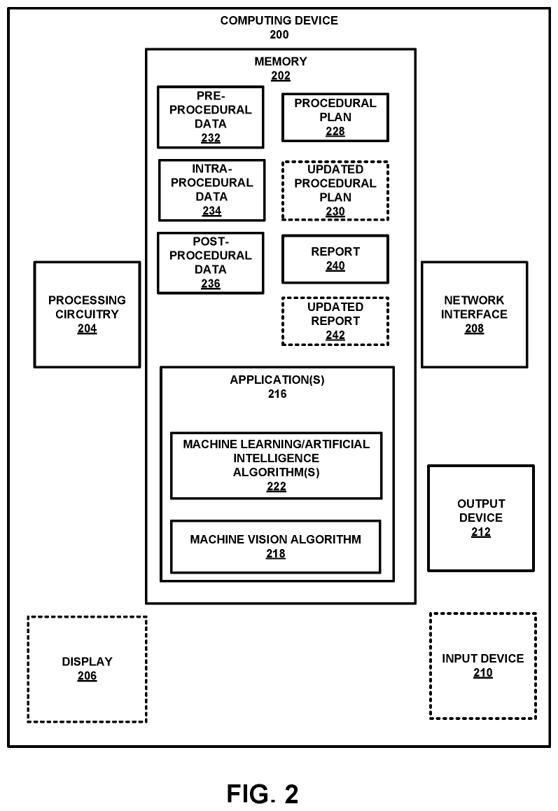

FIG. 2 is a schematic view of one example of a computing device of system 10 of FIG. 1. Computing device 200 may be an example of computing device 150 or server 160 of FIG. 1. Computing device 200 may also be an example of a computing device used to create a procedural plan outside of Cath Lab 100. Computing device 200 may include a workstation, a desktop computer, a laptop computer, a smart phone, a tablet, a server, a dedicated computing device, or any other computing device capable of performing the techniques of this disclosure.

In examples where computing device 200 is used to create the procedural plan and is not located in Cath Lab 100, processing circuitry 204 may control network interface 208 to push or otherwise transmit procedural plan 228 into Cath Lab 100 for use by a clinician during the therapeutic medical procedure. For example, computing device 200 may push procedural plan 228 to guidance workstation 50 and/or computing device 150 in Cath Lab 100. The computing device in Cath Lab 100 may display procedural plan 228 on a display device (e.g., display device 110 and/or display 206 (which may be a part of a user interface)), such as a monitor, an augment reality (AR) or virtual reality (VR) headset, holographs, and/or other display device(s) in Cath Lab 100.

Computing device 200 may be configured to perform processing, control and other functions associated with guidance workstation 50, imager 140, and an optional EM tracking system. Computing device 200 may represent multiple instances of computing devices, each of which may be associated with one or more of guidance workstation 50, imager 140, imager 180, one or more cameras 170, or the EM tracking system. Computing device 200 may include, for example, a memory 202, processing circuitry 204, a display 206, a network interface 208, an input device 210, or an output device 212, each of which may represent any of multiple instances of such a device within the computing system, for ease of description.

While processing circuitry 204 appears in computing device 200 in FIG. 2, in some examples, features attributed to processing circuitry 204 may be performed by processing circuitry of any of computing device 150, server 160, guidance workstation 50, imager 140, imager 180, the EM tracking system, other computing device, or combinations thereof. In some examples, one or more processors associated with processing circuitry 204 in computing system may be distributed and shared across any combination of computing device 150, server 160, guidance workstation 50, imager 140, imager 180, and the EM tracking system. Additionally, in some examples, processing operations or other operations performed by processing circuitry 204 may be performed by one or more processors residing remotely, such as one or more cloud servers or processors, each of which may be considered a part of computing device 200. Computing device 200 may be used to perform any of the methods described in this disclosure, and may form all or part of devices or systems configured to perform such methods, alone or in conjunction with other components, such as components of computing device 150, server 160, guidance workstation 50, imager 140, imager 180, an EM tracking system, or a system including any or all of such systems.

Memory 202 of computing device 200 includes any non-transitory computer-readable storage media for storing data or software that is executable by processing circuitry 204 and that controls the operation of computing device 150, server 160, guidance workstation 50, imager 140, imager 180, or EM tracking system, as applicable. In one or more examples, memory 202 may include one or more solid-state storage devices such as flash memory chips. In one or more examples, memory 202 may include one or more mass storage devices connected to the processing circuitry 204 through a mass storage controller (not shown) and a communications bus (not shown).

Although the description of computer-readable media herein refers to a solid-state storage, it should be appreciated by those skilled in the art that computer-readable storage media may be any available media that may be accessed by the processing circuitry 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium that may be used to store the desired information and that may be accessed by computing device 200. In one or more examples, computer-readable storage media may be stored in the cloud or remote storage and accessed using any suitable technique or techniques through at least one of a wired or wireless connection.

Memory 202 may store pre-procedural data 232, intra-procedural data 234, and post-procedural data 236. Pre-procedural data 232 may include pre-therapeutic imaging data, sensor data (e.g., from a wearable device, implantable device, stethoscope, etc.) and/or patient metadata (e.g., sex, age, weight, height, body mass index, body fat percentage, comorbidities, cholesterol level, blood pressure, blood oxygenation, physical exercise level, heart rate, or the like). Intra-procedural data 234 may include data collected during a therapeutic medical procedure, such as angiography data of the patient undergoing the therapeutic medical procedure, intravascular imaging data of the patient undergoing the therapeutic medical procedure, other imaging data of the patient undergoing the therapeutic medical procedure, echocardiogram data of the patient undergoing the therapeutic medical procedure, sensor data relating to the patient undergoing the therapeutic medical procedure, and/or the like. Post-procedural data 236 may include data collected after a therapeutic medical procedure, such as sensor data relating to the patient, user input data relating to the patient, post-procedural imaging data of the patient (e.g., imaging data generated after the therapeutic medical procedure), physiological data (e.g., FFR or other flow reserve measure) of the patient and/or the like. In some examples, pre-procedural data 232, intra-procedural data 234, and post-procedural data 236 include data related to a plurality of patients and may be used by processing circuitry 204 to train one or more of machine learning/artificial intelligence algorithm(s) 222. In some examples, pre-procedural data 232, intra-procedural data 234, and post-procedural data 236 include data related to a current patient. Processing circuitry 204 may execute a trained machine learning algorithm and/or a trained artificial intelligence algorithm of machine learning/artificial intelligence algorithm(s) 222 to generate procedural plan 228 based on pre-procedural data 232 for the current patient.

Procedural plan 228 may include one or more potential treatments for the current patient. Generally, procedural plan 228 may include one or more of use of a diagnostic catheter, plain old balloon angioplasty (POBA), mechanical atherectomy, intravascular lithotripsy (IVL), drug coated balloon angioplasty, stent delivery (including bare metal stents, drug eluting stents (DES), bioresorbable scaffolds, etc.), post-stenting optimization, wire-based FFR or other flow reserve measure, image-based FFR or other flow reserve measure, OCT, IVUS, etc. As specific examples, for a bifurcation case, a potential treatment could include provisional, T and small protrusion (TAP), inverted provisional, double kissing (DK) culotte, DK crush, etc. For a calcified lesion case, a potential treatment could include lesion crossing, imaging and calcium modification, etc. For a CTO case, a potential treatment could include wire escalation, antegrade, retrograde, dissection & reentry, controlled antegrade and retrograde subintimal tracking (CART), reverse CART, etc. For an ISR case, a potential treatment could include lesion crossing, imaging and lesion treatment, etc. A left main disease case often includes a bifurcation and the bifurcation may be in a last viable vessel feeding the left side of the heart and treatment may include treatment of the bifurcation. Procedural plan 228 may also give the clinician an idea of what medical instruments or devices they may need (e.g., atherectomy, balloons, drug coated balloons, high pressure balloons, cutting or scoring balloons, intravascular lithotripsy (IVL), specialty wires, specialty micro catheters, intravascular imaging, calcium modification tools, stents, drug-eluting stents, mechanical circulation support, etc.) to perform the treatment(s) set forth in the procedural plan.

In some examples, a clinician or a computing device may augment procedural plan 228 via selecting from display 206 and/or input device 210 any or all of the data from Cath Lab 100 in real time. Such data may include invasive angiography, intravascular coronary imaging (e.g., intravascular ultrasound (IVUS), optical coherence tomography (OCT), etc.), echocardiogram (ECG), etc. In some examples, because calcium from a calcified lesion may cause blooming in a 3-dimensional (3D) image, processing circuitry 204 may execute a machine learning algorithm or artificial intelligence algorithm of machine learning/artificial intelligence algorithm(s) 222, such as a neural network, to reduce the blooming in the 3D image from the calcium. For example, the machine learning algorithm or artificial intelligence algorithm may be trained to separate the calcium of the vessel from the native vessel anatomy so as to generate an anatomy only image. For example, the machine learning algorithm or artificial intelligence algorithm may be trained on known ground truths. The ground truths may be created from a library of simulations based upon previous anatomy and from the specific intravascular image provided for a particular case.

In some examples, a clinician may edit procedural plan 228, such as by selecting or substituting one or more proposed treatments, by selecting or substituting one or more preferred medical instruments, or the like, via input device 210.

To determine procedural plan 228, processing circuitry 204 may analyze the coronary issue identified in pre-procedural data 232, e.g., bifurcation lesion, calcified lesion, CTO, ISR, left main disease, etc., and characterize the anatomy, the physiology, morphology, pathology, etc. For example, processing circuitry 204 may execute machine vision algorithm 218 to classify the coronary issue or a clinician can classify the coronary issue through input device 210 or via network interface 208 from another computing device.

For example, in the case of a bifurcation, processing circuitry 204 may analyze the anatomy of the surrounding vasculature of the bifurcation to assist with identifying the specific strategy that could be of use in treating such a case. Processing circuitry 204 may identify and classify the bifurcation disease. For example, processing circuitry 204 may classify the bifurcation disease according to a known classification system, such as a Medina classification and include a 3D image of at least a portion of the vasculature to communicate the severity or condition of the disease to a clinician. For example, some classes of bifurcation disease may respond differently to certain treatments than other classifications of bifurcation disease. Processing circuitry 204 may analyze the bifurcation lesion to identify, for example through performing a plurality of simulations, a strategy for treating the bifurcation lesion. For example, processing circuitry 204 may perform a plurality of simulations using different interventions and select one or more treatments for the PCI having the best simulated patient outcome(s). Processing circuitry 204 may include such one or more treatments in procedural plan 228. Processing circuitry 204 may analyze the anatomy of the vessels of the patient to estimate the position of medical instruments or devices, such as guide wires, microcatheters, balloons, stents, or the like.

In the example of a calcified lesion, processing circuitry 204 may analyze the anatomy of the surrounding vasculature of the calcified lesion to assist with identifying the specific procedural strategy that could be of use in such a case. Processing circuitry 204 may analyze the anatomy of the vessels to estimate the position of medical instruments, such as microcatheters and guide wires, to estimate if adequate support exists to penetrate the calcified lesion. Processing circuitry 204 may analyze the vessel wall characteristics to predict a suitable calcium modification tool or an escalation of medical instruments to be used during the therapeutic medical procedure.

In the case of a CTO, processing circuitry 204 may analyze the distal and proximal cap of the CTO. Processing circuitry 204 may analyze on the CTO to identify any fissures along the lesion that may facilitate the tracking of a guide wire. Processing circuitry 204 may analyze the anatomy of the vessels to estimate the position of medical instruments, such as microcatheters and guide wires, to estimate if adequate support exists to penetrate the patient specific caps. Processing circuitry 204 may analyze the vessel wall characteristics to predict suitability of a dissection and re-entry strategy. Processing circuitry 204 may analyze the vasculature to identify the true lumen for the vessel. This may be useful during the PCI procedure if a guide wire position is uncertain on angiography alone (e.g., it is uncertain whether the guide wire in a true lumen or in a vessel wall). Processing circuitry 204 may analyze the vasculature of the patient to identify a retrograde approach using collaterals or other vessels to permit the medical instrument to travel distal of the lesion.

In the case of an ISR, processing circuitry 204 may analyze the anatomy of the vessels to estimate the position of medical instruments, such as microcatheters and guide wires, to estimate if adequate support exists to penetrate the lesion. Processing circuitry 204 may analyze on the vessel wall characteristics to predict a suitable ISR treatment strategy, including which medical instrument(s) to use. Processing circuitry 204 may analyze the vessel wall to confirm the stent is implanted. In some examples, the confirmation that the sent is implanted may be input manually via input device 210 or pulled into procedural plan 228 from a patient electronic medical record (not shown). Stent design can be used to reduce artifact blooming, as some stents present more artifact blooming than others. Stent design can be used to ensure that the mechanical properties of the stent are accounted for in any subsequent plan or simulation conducted by processing circuitry 204. Processing circuitry 204 may analyze the implanted stent design such as run a simulation on performance of the implanted stent design. Processing circuitry 204 may run simulations on the performance of other stent designs and compare the performance of the implanted stent design against the performance of other stent designs.

In the case of left main disease, processing circuitry 204 may analyze the anatomy of the patient and run simulations to determine the risk level of the therapeutic medical procedure. Processing circuitry 204 may recommend devices such as mechanical circulation support based on the simulation and/or based on the left main disease diagnosis. Processing circuitry 204 may also recommend back up support options such as a hybrid lab heart team in case complications occur during the therapeutic medical procedure. Processing circuitry 204 may analyze the anatomy of the vessels to estimate the position of medical instruments, such as guide wires, microcatheters, balloons, stents, etc., during the therapeutic medical procedure. Processing circuitry 204 may use the simulation having the best outcome to determine one or more treatments to include in procedural plan 228. Processing circuitry 204 may analyze the risk level of the therapeutic medical procedure and then include risk reduction strategies in procedural plan 228. An example of such a risk reduction strategy may include wiring a side branch of a vessel to ensure access to the vessel in case there is a spasm or other response from the vessel. In some examples, processing circuitry 204 or a clinician may request additional information, such as renal function, ejection fraction, LV function, etc., which may influence whether the patient should be protected by mechanical circulatory support. Processing circuitry 204 may use such information to determine whether the patient should be protected by mechanical circulatory support and may include whether the patient should be protected by mechanical circulatory support in the plan. In some examples, processing circuitry 204 may link or connect the clinician with the sales team or representative of the company manufacturing the mechanical circulatory support device, if desired, to ensure a proctor is available for the therapeutic medical procedure. For example, a clinician may not be comfortable using mechanical circulatory support without a proctor from the company. Processing circuitry 204 may indicate if a heart team with a hybrid lab is recommended for back up.

In some examples, processing circuitry 204 may request additional information, such as intravascular imaging to be completed to increase the accuracy of any prediction of success of any of the simulations.

Processing circuitry 204 may determine procedural plan 228 for the PCI, for example, based on one or more of the analyses performed, such as based on the simulations. In some examples, processing circuitry may execute one or more of machine learning/artificial intelligence algorithm(s) or machine vision algorithm 218 when performing such simulations. In some examples, processing circuitry 204 may present a plurality of options for procedural plan 228 via display 206 to a clinician from which the clinician may select via input device 210. In other examples, processing circuitry 204 may determine procedural plan 228 without presenting a plurality of options.

In some examples, procedural plan 228 may include what medical instruments or devices may be used for the therapeutic medical procedure, such as atherectomy, balloons, drug coated balloons, high pressure balloons, cutting or scoring balloons, intravascular lithotripsy (IVL), specialty wires, specialty micro catheters, intravascular imaging, calcium modification tools, stents, drug-eluting stents, mechanical circulation support, etc.

Processing circuitry 204 may execute at least one of a machine learning algorithm or artificial intelligence algorithm (e.g., of machine learning/artificial intelligence algorithm(s) 222) to determine procedural plan 228. For example, the at least one of the machine learning algorithm or artificial intelligence algorithm may be trained using procedural plans, treatments, and outcomes for coronary interventions including data collected pre-procedure, intra-procedure, and post-procedure. Thus, the machine learning algorithm or artificial intelligence algorithm may be trained on actual treatments and actual outcomes from past PCIs and may include treatments in procedural plan 228 based on successful outcomes.

For example, a k-means clustering model may be used having a plurality of clusters: one for each treatment using one or more particular medical instruments and/or devices. Each identified coronary issue may be associated with a vector that includes variables for, e.g., pre-procedural data, intra-procedural data, and post-procedural data, such as type of coronary issue, severity of the coronary issue, complexity of the coronary issue, location of the coronary issue, classification of a lesion, anatomy in the area of the coronary issue, other anatomy, patient metadata (e.g., sex, age, weight, height, body mass index, body fat percentage, comorbidities, cholesterol level, blood pressure, blood oxygenation, physical exercise level, heart rate, etc.), the outcome of the therapeutic medical procedure and/or the like. The location of the vector in a given one of the clusters may be indicative of a particular treatment using one or more particular medical instruments and/or devices. For example, if the vector falls within the cluster for TAP using a particular medical instrument, the machine learning algorithm or the artificial intelligence algorithm may include TAP as a treatment in procedural plan 228 and may include the particular medical instrument in procedural plan 228. Other potential machine learning or artificial intelligence techniques that may be used include Naïve Bayes, k-nearest neighbors, random forest, support vector machines, neural networks, linear regression, logistic regression, etc.

Processing circuitry 204 may determine a specific procedural plan for the patient including one or more treatments. For example, in the case of bifurcation, procedural plan 228 may include any of provisional, TAP, inverted provisional, DK culotte, DK crush, etc. In the case of a calcified lesion, procedural plan 228 may include any of lesion crossing, imaging and calcium modification, etc. In the example of CTO, procedural plan 228 may include any of wire escalation, antegrade, retrograde, dissection & reentry, CART, reverse CART, etc. In the case of ISR, procedural plan 228 may include lesion crossing, imaging and lesion treatment, etc. In the case of left main disease, procedural plan 228 may include any of the treatments set forth above for bifurcation and/or other treatments.

As mentioned above, procedural plan 228 may include which medical instruments and/or devices may be used during the therapeutic medical procedure. In some examples, procedural plan 228 may cross reference medical instruments and/or devices that may be used during the therapeutic medical procedure with inventory available at the facility where the therapeutic medical procedure may be performed, such as a hospital. Procedural plan 228 may also include a cross reference to which medical instruments and/or devices may be approved for use in the region in which Cath Lab 100 is based. Procedural plan 228 may include a step-by-step approach to the therapeutic medical procedure and indicate when and where and how medical instruments and/or devices are to be used. In some examples, procedural plan 228 may include a warning for using particular medical instruments and/or devices in an off-label manner. For example, processing circuitry 204 may cross reference the use case of the devices in procedural plan 228 against the device indications, contraindications, warnings, etc.

During the therapeutic medical procedure, computing device 200 may receive intra-procedural data 234, which may include angiography data of the patient undergoing the therapeutic medical procedure, intravascular imaging data of the patient undergoing the therapeutic medical procedure, other imaging data of the patient undergoing the therapeutic medical procedure, echocardiogram data of the patient undergoing the therapeutic medical procedure, sensor data relating to the patient undergoing the therapeutic medical procedure, and/or the like. Processing circuitry 204 may alter the plan in real time if intra-procedural data indicates that a different treatment plan would provide a better predicted outcome. This may be desirable in complex PCI procedures, as it may increase the likelihood of success or lead to improved patient outcomes. For example, processing circuitry 204 may further execute the trained machine learning algorithm and/or the trained artificial intelligence algorithm of machine learning/artificial intelligence algorithm(s) 222 to determine whether to update procedural plan 228 based on at least one of at least a portion of procedural plan 228 or at least a portion of the intra-procedural data 234 (e.g., based on imaging data). For example, processing circuitry 204 may determine that a different treatment may increase the likelihood of a successful outcome based on at least one of at least a portion of procedural plan 228 and at least a portion of intra-procedural data 234 than a treatment contained within procedural plan 228. As such, processing circuitry 204 may update procedural plan 228 to generate updated procedural plan 230, which processing circuitry 204 may store in memory 202, display via display 206, and/or output via network interface 208 or output device 212. In some examples, processing circuitry 204 may overwrite procedural plan 228 with updated procedural plan 230.

Processing circuitry 204 may further train the trained machine learning algorithm and/or the trained artificial intelligence algorithm of machine learning/artificial intelligence algorithm(s) 222 using pre-procedural data 232, intra-procedural data 234, and post-procedural data 236 of a current patient. For example, in the example of using a k-means clustering model, the k-means clustering model may add the current procedure to the clusters and associate a vector with the pre-procedural data 232, intra-procedural data 234, and post-procedural data 236.

Post-procedural data 236 may include sensor data relating to the patient, user input data relating to the patient, post-procedural imaging data of the patient, physiological data (FFR) of the patient and/or the like. Differences between pre-procedural data 232 and post procedural data 236 may be indicative of the outcome of the therapeutic medical procedure. For example, processing circuitry 204 may determine the differences between pre-procedural data 232 and post procedural data 236 to determine a measure of success of each procedure. For example, if post-procedural imaging data indicates the lesion shown in the pre-procedural data is no longer there, that may be indicative of the success of the procedure. If FFR analysis indicates 95% blood flow after the procedure, but the pre-procedural FFR data indicates a 20% blood flow, that may be indicative of the success of the procedure. If sensor data indicates a patient took many fewer steps and had a higher heart rate prior to the therapeutic medical procedure than after, that may be indicative of some level of success of the therapeutic medical procedure as such data may indicate that the patient's level of physical fitness has improved. The cumulative outcomes for a particular therapeutic medical procedure used to treat a particular coronary issue using a particular medical instrument may provide a measure of likelihood of a successful outcome.

Processing circuitry 204 may be implemented by one or more processors, which may include any number of fixed-function circuits, programmable circuits, or a combination thereof. As described here, guidance workstation 50 may perform various control functions with respect to imager 140 and may interact extensively computing device 200. Guidance workstation 50 may be communicatively coupled to computing device 200, enabling guidance workstation 50 to control the operation of imager 140 and receive the output of imager 140. In some examples, computing device 200 may control various operations of imager 140.

In various examples, control of any function by processing circuitry 204 may be implemented directly or in conjunction with any suitable electronic circuitry appropriate for the specified function. Fixed-function circuits refer to circuits that provide particular functionality and are preset on the operations that may be performed. Programmable circuits refer to circuits that may programmed to perform various tasks and provide flexible functionality in the operations that may be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Instructions may be executed by one or more processors, such as one or more digital signal processors (DSPs), general purpose microprocessors, application specific integrated circuits (ASICs), field programmable gate arrays (FPGAs), graphics processing units (GPUs) or other equivalent integrated or discrete logic circuitry. Accordingly, the term processing circuitry 204 as used herein may refer to one or more processors having any of the foregoing processor or processing structure or any other structure suitable for implementation of the techniques described herein. In addition, in some aspects, the functionality described herein may be provided within dedicated hardware or software modules configured for encoding and decoding, or incorporated in a combined codec. Also, the techniques could be fully implemented in one or more circuits or logic elements.

Display 206 may include a display 206 may be touch sensitive or voice activated, enabling the display to serve as both an input and output device. Alternatively, a keyboard (not shown), mouse (not shown), or other data input devices (e.g., input device 210) may be employed.

Network interface 208 may be adapted to connect to a network such as a local area network (LAN) that includes a wired network or a wireless network, a wide area network (WAN), a wireless mobile network, a Bluetooth network, or the internet. For example, guidance workstation 50, computing device 150, and/or server 160 may receive imaging data from imager 140 and/or imager 180 during a medical procedure via network interface 208. Guidance workstation 50, computing device 150, and/or server 160 may receive updates to its software, for example, application 216, via network interface 208. Guidance workstation 50, computing device 150, and/or server 160 may also display notifications on display 206 that a software update is available.

Input device 210 may be any device that enables a user to interact with guidance workstation 50 and/or computing device 150, such as, for example, a mouse, keyboard, foot pedal, touch screen, augmented-reality input device receiving inputs such as hand gestures or body movements, or voice interface.

Output device 212 may include any connectivity port or bus, such as, for example, parallel ports, serial ports, universal serial busses (USB), or any other similar connectivity port known to those skilled in the art.

Application(s) 216 may be one or more software programs stored in memory 202 and executed by processing circuitry 204 of computing device 150. Processing circuitry 204 may display procedural plan 228, updated procedural plan 230, and/or intra-procedural data 234, for example, during a therapeutic medical procedure, on display 206 and/or display device 110.

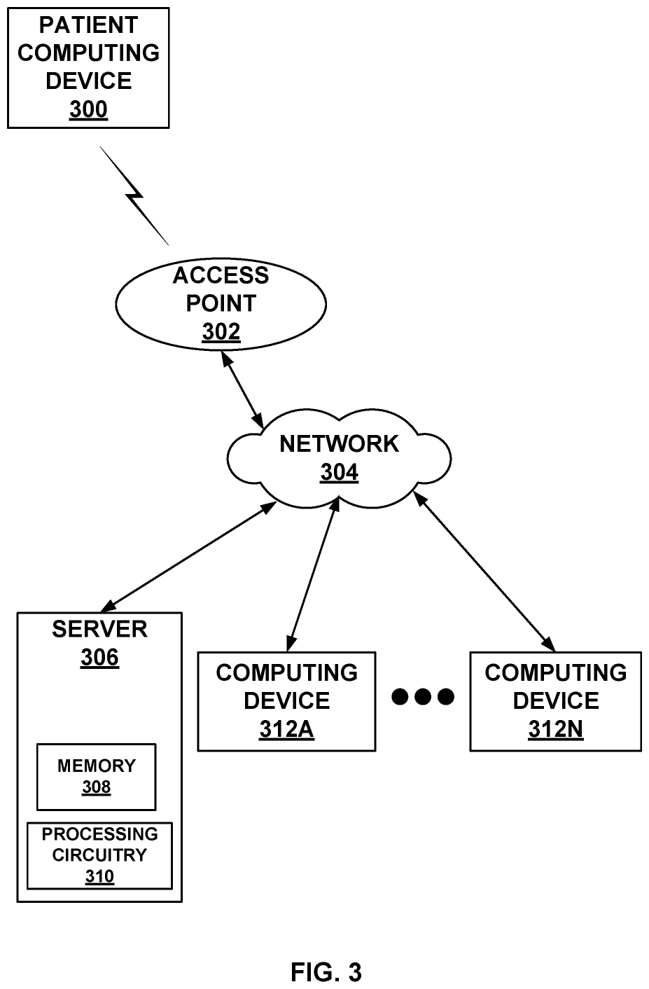

FIG. 3 is a functional block diagram illustrating an example system that includes external computing devices, such as a server and one or more other computing devices that are connected via a network. In the example of FIG. 3, patient computing device 300, access point 302, server 306, and computing devices 312A-312N are interconnected, and able to communicate with each other, through network 304.

Access point 302 may comprise a device that connects to network 304 via any of a variety of connections, such as telephone dial-up, digital subscriber line (DSL), cable modem, or fiber optic connections. In other examples, access point 302 may be coupled to network 304 through different forms of connections, including wired or wireless connections. In some examples, access point 302 may be co-located with the patient. Server 306 may be an example of server 160 of FIG. 1. In some examples, memory 308 may store machine learning/artificial intelligence algorithm(s) 222 and/or machine vision algorithm 218 (FIG. 2) and processing circuitry 310 may execute machine learning/artificial intelligence algorithm(s) 222 and/or machine vision algorithm 218.

Patient computing device and/or computing devices 312A-312N (which may be clinician computing devices) may include a laptop computer, desktop computer, tablet computer, smart phone, or other similar device or may include a specific purpose device. While not shown, in some examples, any or all of computing devices 312A-312N may be connected to network 304 via one or more access points.

In some examples, server 306 may be configured to provide a secure storage site for pre-procedural data 232, intra-procedural data 234, post-procedural data 236, procedural plan 228, updated procedural plan 230, report 240, updated report 242 (all of FIG. 2), any other data related to a patient(s) and/or medical procedure that has been collected. The illustrated system of FIG. 3 may be implemented, in some aspects, with general network technology and functionality similar to that provided by the Medtronic CareLink® Network developed by Medtronic plc, of Dublin, Ireland.

In some examples, one or more of server 306, or computing devices 312 may be configured to perform, e.g., may include processing circuitry configured to perform, some or all of the techniques described herein, e.g., with respect to processing circuitry 204 of computing device 200 (FIG. 2).

In some examples, server 306 may serve as a patient portal, from which a patient may, via patient computing device 300 and access point 302, access their medical information, including information about the medical procedure at a level a lay person could understand. For example, the patient portal may be focused on education and engagement with the patient.

In some examples, one or more of computing devices 312 may be clinician computing devices which may be located at a facility including Cath Lab 100 (FIG. 1) or located elsewhere. In some examples, server 306 may function as a clinician portal, which may store all the collected information regarding the therapeutic medical procedure, including the entire data history, all pre-procedural data 232, all intra-procedural data 234, and all post-procedural data. In some examples, the machine learning algorithm or the artificial intelligence algorithm may be trained on the collected data in the clinician portal, the data collected by each device involved in data collection, or a combination of the two. In some examples, the clinician portal may be specific to coronary artery disease (CAD) identification and treatment strategies. For example, a clinician may access the clinician portal via one of computing devices 312. In some examples, the clinician portal and/or the patient portal may include encryption to provide security from unauthorized access. The clinician portal may include a procedure planner which may employ the techniques disclosed herein.

For example, post-procedural data 236 may include sensor generated data, from, for example, wearable device(s) (such as a smart watch, a patch, or the like) and/or implanted device(s). In some examples, post-procedural data 236 may include data generated by such sensor(s) for thirty days or more. Such data may be sent to patient computing device 300 and be transmitted by patient computing device 300 to the patient portal (e.g., server 306) via access point 302 and network 304. In some examples, all or some of pre-procedural data 232, intra-procedural data 234, and post-procedural data 236 related to a specific patient may be included in patient electronic medical record on server 306 such as to demonstrate the full patient journey value as part of the patient portal or the clinician portal.

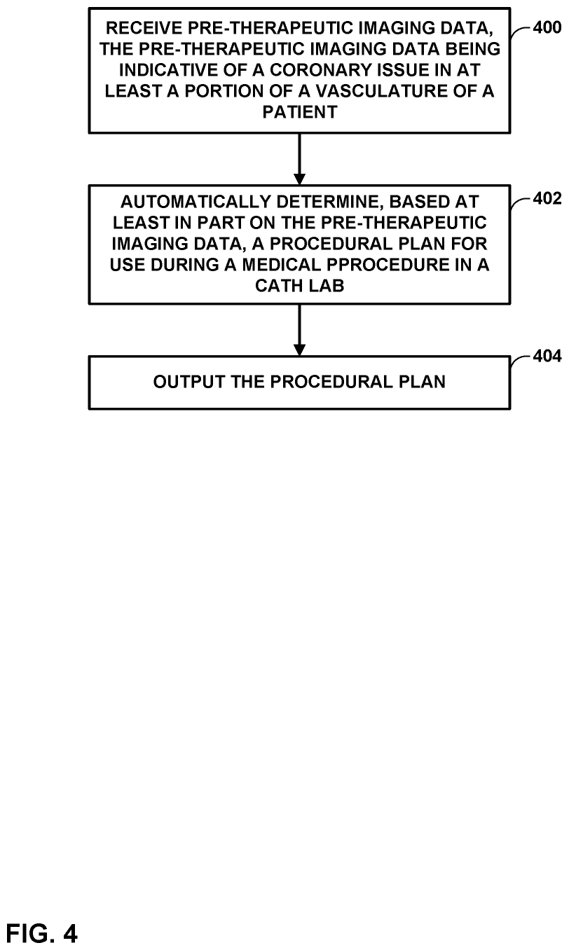

FIG. 4 is a flow diagram of example generation of a procedural plan techniques according to one or more aspects of this disclosure. Processing circuitry 204 may receive pre-therapeutic imaging data, the imaging data being indicative of a coronary issue in at least a portion of a vasculature of a patient (400). For example, processing circuitry 204 may receive pre-procedural data 232 which may include pre-therapeutic imaging data of a patient. Such pre-therapeutic imaging data may have been taken during a diagnostic imaging procedure to assist in the diagnosis of a coronary issue (e.g., before a PCI). The pre-therapeutic imaging data may indicate a coronary issue, such as bifurcation lesions, calcified lesions, CTOs, ISRs, left main disease; etc.

Processing circuitry 204 may automatically determine, based at least in part on the pre-therapeutic imaging data, a procedural plan for use during a therapeutic medical procedure in a Cath Lab (402). For example, processing circuitry 204 may apply at least one of a machine learning algorithm or an artificial intelligence algorithm (of machine learning/artificial intelligence algorithm(s) 222) to the pre-therapeutic imaging data. Additionally, or alternatively, processing circuitry 204 may execute a plurality of simulations of procedures to determine at least one treatment to include the procedural plan.

Processing circuitry 204 may output the procedural plan (404). For example, processing circuitry 204 may output procedural plan 228 to at least one of a computing device (e.g., computing device 150, server 160, computing device 312A, etc.), a user interface (e.g., display 206 or display device 110), or robot 102. For example, a clinician may view procedural plan 228 via display 206 and may use procedural plan 228 to assist in performing the therapeutic medical procedure. A patient or caregiver may view procedural plan 228, or a simplified version of procedural plan 228. Robot 102 may use procedural plan 228 to perform the therapeutic medical procedure. In some examples, such as in a robot assisted medical procedure, both the clinician may view procedural plan 228 and robot 102 may use procedural plan 228 to assist the clinician in performing the therapeutic medical procedure.

In some examples, processing circuitry 204 may receive patient metadata (which may be part of pre-procedural data 232) including at least one of sex, age, weight, height, body mass index, body fat percentage, comorbidities, cholesterol level, blood pressure, blood oxygenation, physical exercise level, or heart rate, and processing circuitry 204 may automatically determine the procedural plan further based on the patient metadata. For example, patient metadata may be imported from a patient electronic medical record, may be input by a clinician, and/or be collected by one or more sensors, such as wearable device, like a smart watch or a fitness watch, a stethoscope, or the like. In some examples, the procedural plan includes at least one of data indicative of one or more treatments, medical instruments to perform the one or more treatments, devices to be used during the one or more treatments, step-by-step indications of how to perform the one or more treatments, indications of when and where and how to use at least one of the medical instruments or devices, or a warning regarding unapproved uses for at least one of the medical instruments or devices. In some examples, the coronary issue includes at least one of a bifurcation lesion, a calcified lesions, a CTO, an ISR, or left main disease.

In some examples, processing circuitry 204 may receive second imaging data (e.g., of intra-procedural data 234) during the therapeutic medical procedure and control a display device (e.g., the display of display 206 or display device 110) to display procedural plan 228 together with the second imaging data during the therapeutic medical procedure. In some examples, processing circuitry 204 may determine, based on at least one of at least a portion of the second imaging data or at least a portion of procedural plan 288, to update procedural plan 288. Processing circuitry 204 may update procedural plan 288 to generate updated procedural plan 230, updated procedural plan 230 including at least one treatment that is not included in procedural plan. Processing circuitry 204 may control the display device to display updated procedural plan 230. For example, processing circuitry 204 may output the updated procedural plan to a computing device (e.g., computing device 150, server 160, computing device 312A, etc.) a display device (e.g., the display of display 206 or display 110), and/or to robot 102.

In some examples, as part of at least one of determining to update the procedural plan or updating the procedural plan, processing circuitry 204 may apply at least one of a machine learning application or an artificial intelligence application (e.g., of machine learning/artificial intelligence algorithm(s) 222) to at least one of at least a portion of the second imaging data or at least a portion of procedural plan 228.

In some examples, processing circuitry 204 may generate report 240 including data collected during the therapeutic medical procedure. In some examples, processing circuitry 204 may update report 240 to generate updated report 242 based on post-procedural data 236 relating to the patient. For example, processing circuitry 204 may make all collected data from the therapeutic medical procedure (all of intra-procedural data 234) available to the clinician via display 206. In some examples, processing circuitry 204 may prepare a summarized report, such as report 240, for the clinician or may facilitate the clinician preparing such a report via display 206. In some examples, display 206 may be configured for the clinician to input outcomes of the PCI, for example, including final pictures of angiography and/or intravascular coronary imaging. In some examples, the clinician may augment the recorded outcomes, for example, in report 240, via display 206, for example, after 30 days or even longer, to create updated report 242. In some examples, the patient may augment the recorded outcome of their PCI procedure via a patient portal or via wearable or implanted sensors. Processing circuitry may include the captured data of the PCI procedure, the plan, the actual treatment, the recorded outcome, and/or the augmented outcome in the patient medical record. Processing circuitry may control telemetry circuitry to push or otherwise transmit the data to one or more devices that may execute the machine learning algorithm or artificial intelligence algorithm to be used to further train the machine learning algorithm or artificial intelligence algorithm. For example, the one or more devices may be located in the facility or in a cloud-based computing network. In this manner, the machine learning algorithm or artificial intelligence algorithm may be improved for developing procedural plans for a therapeutic medical procedure using pre-therapeutic imaging.

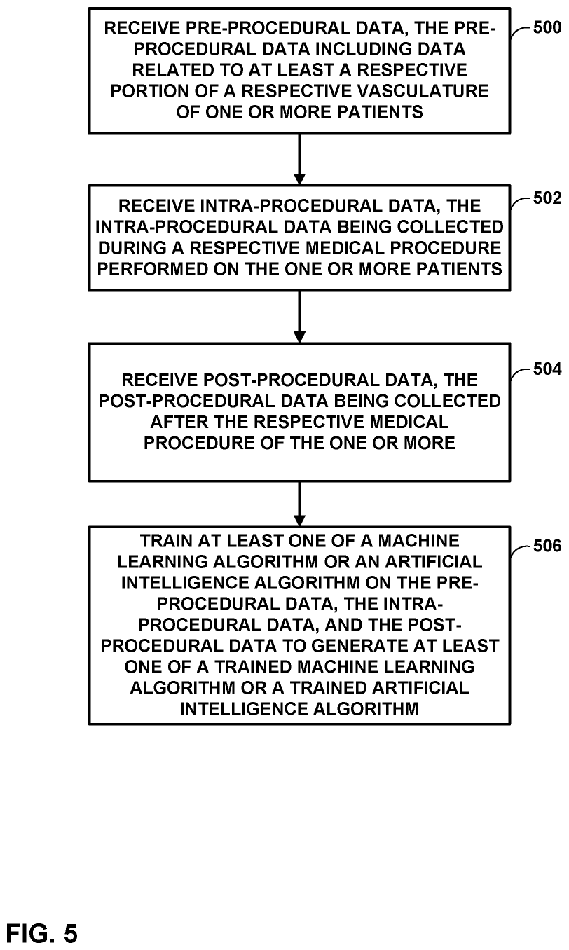

FIG. 5 is a flow diagram of example machine learning algorithm or artificial intelligence algorithm training techniques according to one or more aspects of this disclosure. Processing circuitry 204 may receive pre-procedural data 232 (500). For example, processing circuitry 204 may receive pre-procedural data 232 from diagnostic imaging system(s) (not shown), wearable device(s) not shown, server 160, patient electronic medical records, clinician input, or the like. Pre-procedural data 232 may include data related to at least a respective portion of a respective vasculature of one or more patients. In some examples, pre-procedural data 232 includes at least one of pre-therapeutic imaging data of at least a respective portion of a respective vasculature of the one or more patients or sensor data relating to the one or more patients, such as sensor data collected by a wearable device, like a smart watch or a fitness watch, a stethoscope, or the like. In some examples, pre-procedural data 232 may include patient metadata such as sex, age, weight, height, body mass index, body fat percentage, comorbidities, cholesterol level, blood pressure, blood oxygenation, physical exercise level, or heart rate.

Processing circuitry 204 may receive intra-procedural data 234, intra-procedural data 234 being collected during a respective therapeutic medical procedure performed on the one or more patients (502). For example, processing circuitry 204 may receive intra-procedural data 234 from imager 140, imager 180, one or more video cameras 190, and/or the like, in real-time while the therapeutic medical procedure is being conducted. In some examples, intra-procedural data 234 includes at least one of angiography data of the one or more patients, intravascular imaging data or the one or more patients, echocardiogram data of the one or more patients, sensor data of the one or more patients, or video data. In some examples, the video data includes indications of at least one of hand movements, robot movements, medical instruments or devices used, when medical instruments or devices are used, or where medical instruments or devices are used.

Processing circuitry 204 may receive post-procedural data 236, post-procedural data 236 being collected after the respective therapeutic medical procedure of the one or more patients (504). For example, processing circuitry 204 may receive post-procedural data 236 after the respective therapeutic medical procedure is completed from, for example, a wearable device, a diagnostic imaging system, a user interface, an FFR device, server 160, and/or the like. In some examples, the post-procedural data includes at least one of post-procedural sensor data relating to the one or more patients, user input data relating to the one or more patients, post-procedural imaging data of the one or more patients, or physiological data of the one or more patients.

Processing circuitry 204 may train at least one of a machine learning algorithm or an artificial intelligence algorithm (e.g., of machine learning/artificial intelligence algorithm(s) 222) on pre-procedural data 232, intra-procedural data 234, and post-procedural data 236 to generate at least one of a trained machine learning algorithm or a trained artificial intelligence algorithm (e.g., of machine learning/artificial intelligence algorithm(s) 222) (506). For example, processing circuitry 204 may input pre-procedural data 232, intra-procedural data 234, and post-procedural data 236 into the machine learning algorithm and/or the artificial intelligence algorithm to train the machine learning algorithm and/or the artificial intelligence algorithm.

In some examples, at least a portion of system 10 is based in a cloud computing environment. In some examples, the one or more patients include a current patient. In some examples, processing circuitry 204 may receive current pre-procedural data (e.g., of pre-procedural data 232) for the current patient. Processing circuitry 204 may apply at least one of the trained machine learning algorithm or the trained artificial intelligence algorithm (e.g., of machine learning/artificial intelligence algorithm(s) 222) to the current pre-procedural data for the current patient. Processing circuitry 204 may automatically determine, based on the application of the trained machine learning algorithm or the trained artificial intelligence algorithm to the current pre-procedural data for the current patient, procedural plan 228 for the current patient and output procedural plan 228 for the current patient to be used during a therapeutic medical procedure.

In some examples, processing circuitry 204 may receive current intra-procedural data (e.g., of intra-procedural data 234) for the current patient. Processing circuitry 204 may apply at least one of the trained machine learning algorithm or the trained artificial intelligence algorithm to at least one of at least a portion of the current intra-procedural data or at least a portion of procedural plan 228 for the current patient. Processing circuitry 204 may determine, based on the application of the trained machine learning algorithm or the trained artificial intelligence algorithm to at least one of the at least a portion of the current intra-procedural data for the current patient or the at least a portion of procedural plan 228 for the current patient, to update procedural plan 228. Processing circuitry 204 may update procedural plan 228 to generate an updated procedural plan 230 and output updated procedural plan 230 for the current patient for use during the therapeutic medical procedure.

In some examples, as part of determining to update procedural plan 230, processing circuitry 204 may determine that a second treatment that is not included in procedural plan 228 has a higher likelihood of successful patient outcome than a first treatment that is included in procedural plan 228 and wherein updated procedural plan 230 includes the second treatment. In some examples, processing circuitry 204 may output procedural plan 228 to at least one of a computing device, display 206, or robot 102.

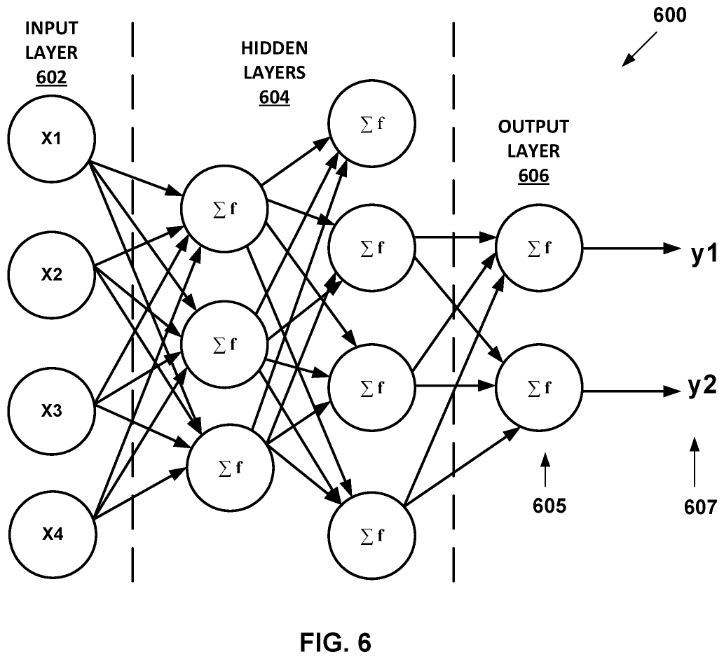

FIG. 6 is a conceptual diagram illustrating an example machine learning model according to one or more aspects of this disclosure. Machine learning model 600 may be an example of the machine learning/artificial intelligence algorithm(s) 222. In some examples, machine learning model 600 may be a part of machine vision algorithm 218 discussed above with respect to FIG. 2. Machine learning model 600 may be an example of a deep learning model, or deep learning algorithm, trained to determine a patient condition and/or a type of medical procedure. One or more of computing device 150 and/or server 160 may train, store, and/or utilize machine learning model 600, but other devices of system 10 may apply inputs to machine learning model 600 in some examples. In some examples, other types of machine learning and deep learning models or algorithms may be utilized in other examples. For examples, a convolutional neural network model of ResNet-18 may be used. Some non-limiting examples of models that may be used for transfer learning include AlexNet, VGGNet, GoogleNet, ResNet50, or DenseNet, etc. Some non-limiting examples of machine learning techniques include Support Vector Machines, K-Nearest Neighbor algorithm, and Multi-layer Perceptron.

As shown in the example of FIG. 6, machine learning model 600 may include three types of layers. These three types of layers include input layer 602, hidden layers 604, and output layer 606. Output layer 606 comprises the output from the transfer function 605 of output layer 606. Input layer 602 represents each of the input values X1 through X4 provided to machine learning model 600. In some examples, the input values may include any of the of values input into the machine learning model, as described above. For example, the input values may include pre-procedural data 232, intra-procedural data 234 and/or post-procedural data 236, as described above. In addition, in some examples input values of machine learning model 600 may include additional data, such as other data that may be collected by or stored in system 10.

Each of the input values for each node in the input layer 602 is provided to each node of a first layer of hidden layers 604. In the example of FIG. 6, hidden layers 604 include two layers, one layer having four nodes and the other layer having three nodes, but fewer or greater number of nodes may be used in other examples. Each input from input layer 602 is multiplied by a weight and then summed at each node of hidden layers 604. During training of machine learning model 600, the weights for each input are adjusted to establish the relationship between pre-procedural data 232, intra-procedural data 234 and/or post-procedural data 236 and a procedural plan (e.g., procedural plan 228 and/or updated procedural plan 230). In some examples, one hidden layer may be incorporated into machine learning model 600, or three or more hidden layers may be incorporated into machine learning model 600, where each layer includes the same or different number of nodes.

The result of each node within hidden layers 604 is applied to the transfer function of output layer 606. The transfer function may be liner or non-linear, depending on the number of layers within machine learning model 600. Example non-linear transfer functions may be a sigmoid function or a rectifier function. The output 607 of the transfer function may be a classification that pre-procedural data 232, intra-procedural data 234 and/or post-procedural data 236 is indicative of a particular procedural plan (e.g., procedural plan 228 and/or updated procedural plan 230).

As shown in the example above, by applying machine learning model 600 to input data such as pre-procedural data 232, intra-procedural data 234 and/or post-procedural data 236, processing circuitry 204 is able to generate and/or update a procedural plan. This may improve patient outcomes.

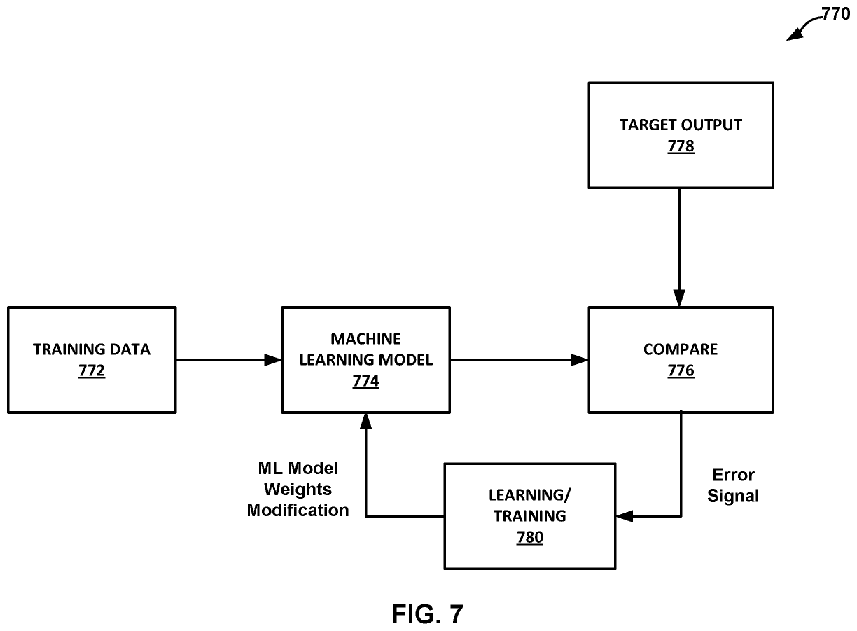

FIG. 7 is a conceptual diagram illustrating an example training process for a machine learning model according to one or more aspects of this disclosure. Process 700 may be used to train machine learning/artificial intelligence algorithm(s) 222 and/or machine vision algorithm 218. A machine learning model 774 (which may be an example of machine learning model 600 and/or machine learning/artificial intelligence algorithm(s) 222) may be implemented using any number of models for supervised and/or reinforcement learning, such as but not limited to, an artificial neural network, a decision tree, naïve Bayes network, support vector machine, or k-nearest neighbor model, CNN, RNN, LSTM, ensemble network, to name only a few examples. In some examples, one or more of computing device 150 and/or server 160 initially trains machine learning model 774 based on a corpus of training data 772. Training data 772 may include, for example, pre-procedural data 232, intra-procedural data 234 and/or post-procedural data 236, other training data which may be mentioned herein, and/or the like.

While training machine learning model 774, processing circuitry of system 2 may compare 776 a prediction or classification with a target output 778. Processing circuitry 204 may utilize an error signal from the comparison to train (learning/training 780) machine learning model 774. Processing circuitry 204 may generate machine learning model weights or other modifications which processing circuitry 204 may use to modify machine learning model 774. For examples, processing circuitry 204 may modify the weights of machine learning model 600 based on the learning/training 480. For example, one or more of computing device 150 and/or server 160, may, for each training instance in training data 772, modify, based on training data 772, the manner in which a procedural plan is generated and/or updated.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The terms “controller”, “processor”, or “processing circuitry” may generally refer to any of the foregoing logic circuitry, alone or in combination with other logic circuitry, or any other equivalent circuitry. A control unit comprising hardware may also perform one or more of the techniques of this disclosure. Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), or electronically erasable programmable read only memory (EEPROM), or other computer readable media.

This disclosure includes the following non-limiting examples.

Example 1. A medical system comprising: memory configured to store one or more procedural plans; and processing circuitry communicatively coupled to the memory, the processing circuitry being configured to: receive pre-therapeutic imaging data, the pre-therapeutic imaging data being indicative of a coronary issue in at least a portion of a vasculature of a patient; automatically determine, based at least in part on the pre-therapeutic imaging data, a procedural plan for use during a therapeutic medical procedure in a Catheterization Laboratory; and output the procedural plan.

Example 2. The medical system of example 1, wherein the processing circuitry is further configured to receive patient metadata comprising at least one of sex, age, weight, height, body mass index, body fat percentage, comorbidities, cholesterol level, blood pressure, blood oxygenation, physical exercise level, or heart rate, and wherein the processing circuitry automatically determines the procedural plan further based on the patient metadata.

Example 3. The medical system of example 1 or example 2, wherein the procedural plan comprises at least one of data indicative of one or more treatments, medical instruments to perform the one or more treatments, devices to be used during the one or more treatments, step-by-step indications of how to perform the one or more treatments, indications of when and where and how to use at least one of the medical instruments or devices, or a warning regarding unapproved uses for at least one of the medical instruments or devices.

Example 4. The medical system of any of examples 1-3, wherein as part of determining the procedural plan, the processing circuitry is configured to apply at least one of a machine learning algorithm or an artificial intelligence algorithm to the pre-therapeutic imaging data.

Example 5. The medical system of any of examples 1-4, wherein as part of determining the procedural plan, the processing circuitry is configured to execute a plurality of simulations of procedures to determine at least one treatment to include the procedural plan.

Example 6. The medical system of any of examples 1-5, wherein the coronary issue comprises at least one of a bifurcation lesion, a calcified lesions, a chronic total occlusion (CTO), an in-stent restenosis (ISR), or left main disease.

Example 7. The medical system of any of examples 1-6, wherein the processing circuitry is configured to output the procedural plan to at least one of a computing device, a user interface, or a robot.

Example 8. The medical system of any of examples 1-7, wherein the processing circuitry is further configured to: receive second imaging data during the therapeutic medical procedure; and control a display device to display the procedural plan together with the second imaging data during the therapeutic medical procedure.

Example 9. The medical system of example 8, wherein the processing circuitry is further configured to: determine, based on at least one of at least a portion of the second imaging data or at least a portion of the procedural plan, to update the procedural plan; update the procedural plan to generate an updated procedural plan, the updated procedural plan comprising at least one treatment that is not included in the procedural plan; and control the display device to display the updated procedural plan.

Example 10. The medical system of example 9, wherein as part of at least one of determining to update the procedural plan or updating the procedural plan, the processing circuitry is configured to apply at least one of a machine learning application or an artificial intelligence application to at least one of at least a portion of the second imaging data or at least a portion of the procedural plan.

Example 11. The medical system of any of examples 1-10, wherein the processing circuitry is further configured to generate a report comprising data collected during the therapeutic medical procedure.

Example 12. The medical system of example 11, wherein the processing circuitry is further configured to update the report to generate an updated report based on post-procedural data relating to the patient.

Example 13. A method comprising: receiving, by processing circuitry, procedural pre-therapeutic imaging data, the pre-therapeutic imaging data being indicative of a coronary issue in at least a portion of a vasculature of a patient; automatically determining, by the processing circuitry and based at least in part on the pre-therapeutic imaging data, a procedural plan for use during a therapeutic medical procedure in a Catheterization Laboratory; and outputting, by the processing circuitry, the procedural plan.

Example 14. The method of example 13, further comprising receiving, by the processing circuitry, patient metadata comprising at least one of sex, age, weight, height, body mass index, body fat percentage, comorbidities, cholesterol level, blood pressure, blood oxygenation, physical exercise level, or heart rate, and wherein automatically determining the procedural plan is further based on the patient metadata.

Example 15. The method of example 13 or example 14, wherein the procedural plan comprises at least one of data indicative of one or more treatments, medical instruments to perform the one or more treatments, devices to be used during the one or more treatments, step-by-step indications of how to perform the one or more treatments, indications of when and where and how to use at least one of the medical instruments or devices, or a warning regarding unapproved uses for at least one of the medical instruments or devices.

Example 16. The method of any of examples 13-15, wherein determining the procedural plan comprises applying at least one of a machine learning algorithm or an artificial intelligence algorithm to the pre-therapeutic imaging data.

Example 17. The method of any of examples 13-16, wherein determining the procedural plan comprises executing a plurality of simulations of procedures to determine at least one treatment to include the procedural plan.

Example 18. The method of any of examples 13-17, wherein the coronary issue comprises at least one of a bifurcation lesion, a calcified lesions, a chronic total occlusion (CTO), an in-stent restenosis (ISR), or left main disease.

Example 19. The method of any of examples 13-18, wherein outputting the procedural plan comprises outputting the procedural plan to at least one of a computing device, a user interface, or a robot.

Example 20. The method of any of examples 13-19, further comprising: receiving, by the processing circuitry, second imaging data during the therapeutic medical procedure; and controlling, by the processing circuitry, a display device to display the procedural plan together with the second imaging data during the therapeutic medical procedure.

Example 21. The method of example 20, further comprising: determining, by the processing circuitry, based on at least one of at least a portion of the second imaging data or at least a portion of the procedural plan, to update the procedural plan; updating, by the processing circuitry, the procedural plan to generate an updated procedural plan, the updated procedural plan comprising at least one treatment that is not included in the procedural plan; and control, by the processing circuitry, the display device to display the updated procedural plan.

Example 22. The method of example 21, wherein at least one of determining to update the procedural plan or updating the procedural plan comprises applying a machine learning application or an artificial intelligence application to at least one of at least a portion of the second imaging data or at least a portion of the procedural plan.

Example 23. The method of any of examples 13-22, further comprising generating a report comprising data collected during the therapeutic medical procedure.

Example 24. The method of example 23, further comprising updating the report to generate an updated report based on post-procedural data relating to the patient.

Example 25. A non-transitory computer-readable storage medium storing instructions, which when executed, cause processing circuitry to: receive pre-therapeutic imaging data, the pre-therapeutic imaging data being indicative of a coronary issue in at least a portion of a vasculature of a patient; automatically determine, based at least in part on the pre-therapeutic imaging data, a procedural plan for use during a therapeutic medical procedure in a Catheterization Laboratory; and output the procedural plan.

Various examples have been described. These and other examples are within the scope of the following claims.

1. A medical system comprising:
memory configured to store one or more procedural plans; and
processing circuitry communicatively coupled to the memory, the processing circuitry being configured to:
receive pre-therapeutic imaging data, the pre-therapeutic imaging data being indicative of a coronary issue in at least a portion of a vasculature of a patient;
automatically determine, based at least in part on the pre-therapeutic imaging data, a procedural plan for use during a therapeutic medical procedure in a Catheterization Laboratory; and
output the procedural plan.

2. The medical system of claim 1, wherein the processing circuitry is further configured to receive patient metadata comprising at least one of sex, age, weight, height, body mass index, body fat percentage, comorbidities, cholesterol level, blood pressure, blood oxygenation, physical exercise level, or heart rate, and wherein the processing circuitry automatically determines the procedural plan further based on the patient metadata.

3. The medical system of claim 1, wherein the procedural plan comprises at least one of data indicative of one or more treatments, medical instruments to perform the one or more treatments, devices to be used during the one or more treatments, step-by-step indications of how to perform the one or more treatments, indications of when and where and how to use at least one of the medical instruments or devices, or a warning regarding unapproved uses for at least one of the medical instruments or devices.

4. The medical system of claim 1, wherein as part of determining the procedural plan, the processing circuitry is configured to apply at least one of a machine learning algorithm or an artificial intelligence algorithm to the pre-therapeutic imaging data.

5. The medical system of claim 1, wherein as part of determining the procedural plan, the processing circuitry is configured to execute a plurality of simulations of procedures to determine at least one treatment to include the procedural plan.

6. The medical system of claim 1, wherein the coronary issue comprises at least one of a bifurcation lesion, a calcified lesions, a chronic total occlusion (CTO), an in-stent restenosis (ISR), or left main disease.

7. The medical system of claim 1, wherein the processing circuitry is configured to output the procedural plan to at least one of a computing device, a user interface, or a robot.

8. The medical system of claim 1, wherein the processing circuitry is further configured to:
receive second imaging data during the therapeutic medical procedure; and
control a display device to display the procedural plan together with the second imaging data during the therapeutic medical procedure.

9. The medical system of claim 8, wherein the processing circuitry is further configured to:
determine, based on at least one of at least a portion of the second imaging data or at least a portion of the procedural plan, to update the procedural plan;
update the procedural plan to generate an updated procedural plan, the updated procedural plan comprising at least one treatment that is not included in the procedural plan; and
control the display device to display the updated procedural plan.

10. The medical system of claim 9, wherein as part of at least one of determining to update the procedural plan or updating the procedural plan, the processing circuitry is configured to apply at least one of a machine learning application or an artificial intelligence application to at least one of at least a portion of the second imaging data or at least a portion of the procedural plan.

11. The medical system of claim 1, wherein the processing circuitry is further configured to generate a report comprising data collected during the therapeutic medical procedure.

12. The medical system of claim 11, wherein the processing circuitry is further configured to update the report to generate an updated report based on post-procedural data relating to the patient.

13. A method comprising:
receiving, by processing circuitry, procedural pre-therapeutic imaging data, the pre-therapeutic imaging data being indicative of a coronary issue in at least a portion of a vasculature of a patient;
automatically determining, by the processing circuitry and based at least in part on the pre-therapeutic imaging data, a procedural plan for use during a therapeutic medical procedure in a Catheterization Laboratory; and
outputting, by the processing circuitry, the procedural plan.

14. The method of claim 13, further comprising receiving, by the processing circuitry, patient metadata comprising at least one of sex, age, weight, height, body mass index, body fat percentage, comorbidities, cholesterol level, blood pressure, blood oxygenation, physical exercise level, or heart rate, and wherein automatically determining the procedural plan is further based on the patient metadata.

15. A non-transitory computer-readable storage medium storing instructions, which when executed, cause processing circuitry to:
receive pre-therapeutic imaging data, the pre-therapeutic imaging data being indicative of a coronary issue in at least a portion of a vasculature of a patient;
automatically determine, based at least in part on the pre-therapeutic imaging data, a procedural plan for use during a therapeutic medical procedure in a Catheterization Laboratory; and
output the procedural plan.

16. The method of claim 13, wherein the procedural plan comprises at least one of data indicative of one or more treatments, medical instruments to perform the one or more treatments, devices to be used during the one or more treatments, step-by-step indications of how to perform the one or more treatments, indications of when and where and how to use at least one of the medical instruments or devices, or a warning regarding unapproved uses for at least one of the medical instruments or devices.

17. The method of claim 13, wherein determining the procedural plan comprises applying at least one of a machine learning algorithm or an artificial intelligence algorithm to the pre-therapeutic imaging data.

18. The method of claim 13, wherein determining the procedural plan comprises executing a plurality of simulations of procedures to determine at least one treatment to include the procedural plan.

19. The method of claim 13, wherein the coronary issue comprises at least one of a bifurcation lesion, a calcified lesions, a chronic total occlusion (CTO), an in-stent restenosis (ISR), or left main disease.

20. The method of claim 13, wherein outputting the procedural plan comprises outputting the procedural plan to at least one of a computing device, a user interface, or a robot.
